(19) United States
(12) Patent Application Publication
(10) Pub. No.: US 2021/0308977 A1
(43) Pub. Date: Oct. 7, 2021

(54) METHOD FOR GENERATING SCENT-FILLED INFLATED AIR CUSHIONS AND WEB OF UNINFLATED SCENTED AIR CUSHIONS

(71) Applicant: Scentisphere LLC, Carmel, NY (US)

(72) Inventor: James Berard, Pleasantville, NY (US)

(21) Appl. No.: 16/841,553

(22) Filed: Apr. 6, 2020

(51) Int. Cl. B31D 5/00; B29C 65/00; A61L 9/012

(52) U.S. Cl. CPC B31D 5/0078; A61L 9/012; B29C 66/439

(57) ABSTRACT

A web of uninflated scented air pillows and a method of forming an air pillow filled with scented air. A scented material is applied to a first surface of a sheet of film material. The sheet of film material is formed into a two-ply web of film material with the first surface positioned as an inner surface within that web. A series of uninflated air chambers are formed in the two-ply web of film material, each of the uninflated air chambers bounded transversely and at a bottom edge and having an open upper longitudinal edge. In this manner, each of uninflated air chambers has scented material on an interior surface thereof. Each uninflated air chambers can be inflated and sealed to form an air pillow. The scented material on the inner surface of the inflated air chambers evaporates upon filling and the air within each inflated air pillow becomes scented.

### FIELD

This disclosure relates generally to a method for generating scent-filled inflated air cushions and a web of uninflated scented air cushions.

### BACKGROUND

There have been many techniques used to pack goods into containers for shipping and many shipping protection materials have been used to absorb impacts on goods packed for shipping. Example shipping protection materials include, inter alia, packing peanuts, molded components, formed paper, and molded pulp packaging components. Air-inflated cushions (or “air pillows”) formed from a film material have also been used to protect goods packed into boxes for shipping. Air pillows offer the lightest weight solution for protective packaging and are durable enough to provide excellent product protection in minimal space. This style of packaging allows uninflated air pillow film materials to be shipped to packers in smaller volume packaging. The uninflated air cushion film materials may be formed into a web that is shipped as a roll or in fan-folded form. The air pillows are then filled as needed by the packer using a standard air pillow inflating machine to form a shock-absorbing packing material (i.e., an air pillow or group of air pillows). Specialized air pillow inflating machines have been created which insert scented air into each air pillow as it is inflated. These specialized air pillow inflating machines are more costly than standard air pillow inflating machines and require scent additives (e.g., fluids or gels) to generate the scented air in each air pillow.

Accordingly, there is a need for to a method for generating scent-filled inflated air cushions and a web of uninflated scented air cushions which do not require specialized machinery or a supply of scent additives.

### BRIEF DESCRIPTION OF THE DRAWINGS

The following detailed description, given by way of example and not intended to limit the present disclosure solely thereto, will best be understood in conjunction with the accompanying drawings in which:

FIG. 1 is a diagram of a web-printer for use according to the method of the present disclosure;

FIG. 2A is a drawing of a portion of an unformed web according to an embodiment of the present disclosure;

FIG. 2B is a drawing of a portion of an unformed web according to another embodiment of the present disclosure;

FIG. 3A is a drawing of a portion of a formed uninflated web according to an embodiment of the present disclosure;

FIG. 3B is a drawing of a portion of a formed uninflated web according to another embodiment of the present disclosure; and

FIG. 4 is a flowchart showing the operation of the method of the present disclosure.

### DETAILED DESCRIPTION

In the present disclosure, like reference numbers refer to like elements throughout the drawings, which illustrate various exemplary embodiments of the present disclosure.

Generally, air pillows (cushioning products) are formed from a web of film materials having two layers joined together by heat seals, the heat seals forming a sequential series of inflatable chambers. The layers may be formed from separate films mated together or from a single folded-over layer. Each inflatable chamber may be separately inflated with air and sealed to form an air pillow, each sequential air pillow separated from each other air pillow by perforations in the transverse heat seal separating each sequential inflatable chamber.

Air pillows are typically manufactured using a web of sheet material such as a flexible thermoplastic film. The web of sheet material is typically provided in a rolls. The film may have advertising or other relevant information printed on a surface thereof prior to formation of the air pillows, this printed surface becoming an outer surface of the air pillows after formation thereof. In accordance with the present disclosure, a scented material is applied to a surface of the film that becomes an inner surface upon formation of the air pillows. The scented material may be an ink that includes an odor-releasing additive such as a fragrant oil that is printed onto the surface of the film using a conventional film printer. For the purpose of this disclosure, the work “ink” is broadly defined as any material that can be applied to a film using a conventional film printer and does not require pigments or other elements required to bind the ink to the surface of the film. The scented material may be applied in other ways to the surface of the film as well. For example, the scented material may be extruded in-line using a nozzle in the same manner that cold glue is applied to printed matter. After application of the scented material (e.g., a scented ink), the film is formed into a web of uninflated air pillows. Then, once each uninflated air pillow is inflated using a conventional air pillow inflating machine, the scented material on the interior surface of each air pillow evaporates and causes the air within the interior of that air pillow to become scented with the particular fragrance included in the scented material. By applying the scented material to the film before formation of the web of uninflated pillows (e.g., by preprinting the film with scented ink using a conventional printer), the method of the present disclosure is able to provide scented air pillows without the need for specialized machinery or a supply of scent additives at the end user's location.

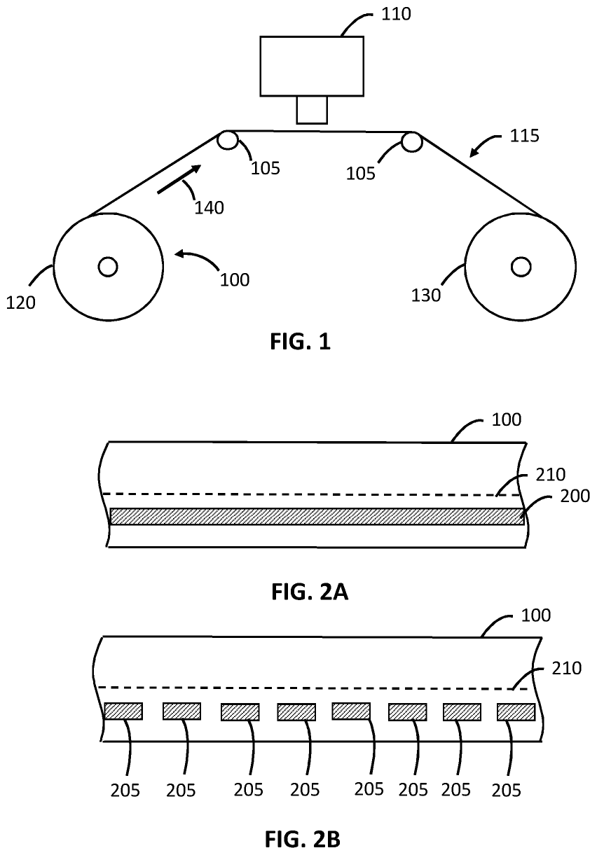

The process of printing the film with a scented ink is shown in FIG. 1. As discussed above, there are various ways to deposit a scented material onto the film and all of the other ways known to those of skill in the art are to be included within the scope of the present disclosure. In particular, a roll of sheet material 100 in FIG. 1 (the film used to form the web of uninflated web pillows) is fed through a system of rollers 105 to a printer 110 to be printed on a selected surface 115 of the sheet material (i.e., the surface which will become the inner surface of the web of uninflated air pillows after formation thereof). The roll 100 is fed from a first roll 120 to a second roll 130 as shown by arrow 140. In accordance with the method of the present disclosure, printer 110 prints a scented ink, i.e., an ink including an odor-releasing additive such as a fragrant oil, on the selected surface 115 of the sheet material 100. Printer 110 may also print other information onto the same surface while printing the scented ink or may print other information on the opposite surface of the sheet material during a second printing operation.

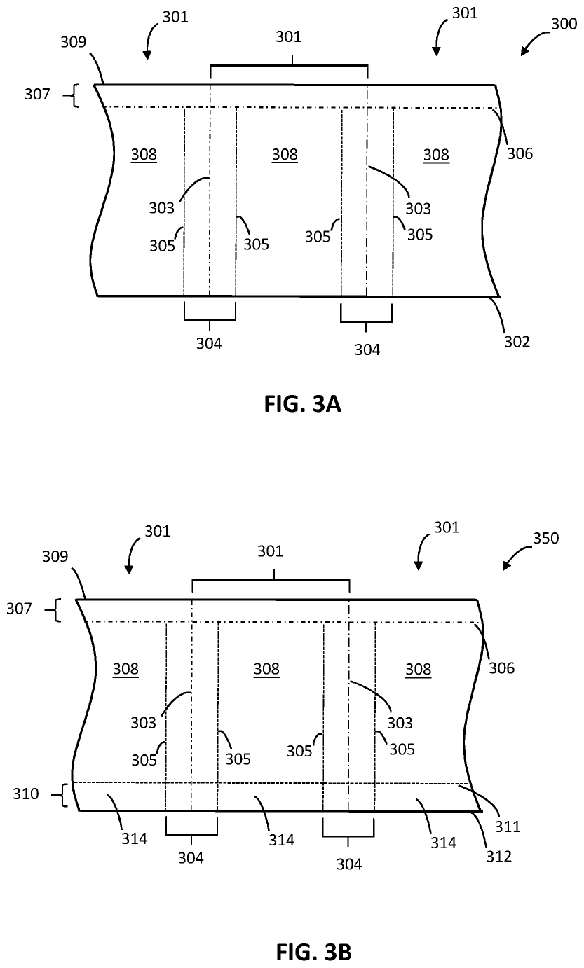

As shown in FIG. 2A, the scented ink (or other scented material) may be applied in a continuous pattern 200 along a longitudinal length of the sheet material 100. Alternatively, as shown in FIG. 2B, the scented ink (or other scented material) may be applied in discontinuous pattern including a series of elements 205 along a longitudinal length of the sheet material 100, each of the elements 205 is positioned to be on an interior wall of each formed air cavity 308 (FIGS. 3A and 3B) of each uninflated air pillow after formation thereof (the spaces between each of the discontinuous pattern elements 205 corresponding to the respective heat seals 304 shown in FIGS. 3A and 3B). The height and characters (or droplet size, etc.) for continuous pattern 200 and discontinuous pattern elements 205 are selected to ensure that an appropriate amount scented ink appears on an interior wall of each formed air cavity 308 (FIGS. 3A and 3B) to ensure that a desired level of scent is released into each air pillow upon formation thereof. The continuous line 200 in FIG. 2A and the discontinuous pattern elements 205 in FIG. 2B are shown below dotted line 210, which represents the fold line for the case where the web of uninflated air pillows is formed from a single roll of sheet material. As discussed above, the actual shape in which the scented ink is printed (e.g., type of character and height) or otherwise applied on the surface of the sheet material 100 is chosen to ensure that an adequate amount of scented ink appears on the interior walls of each uninflated air pillow to ensure that the air inserted during formation of each air pillow becomes scented to a desired amount.

Referring to FIG. 3A, a portion of an uninflated web 300 is shown after formation of the uninflated air pillows using a single roll of sheet material. Each uninflated air pillow 301 is formed from a single film folded longitudinally into two layers at a fold 302 to form a two-ply web, the open ends mating at upper edge 309. Transverse heat seals 304 extend from fold 302 to a line 306 mating the two sides (folded portions) of the sheet material. Each transverse heat seal 304 is bounded laterally by lines 305, forming air chambers 308 for each air pillow 301. Each transverse heat seal 304 extends only from fold 302 to line 306, forming a region 307 extending along the entire upper edge 309 which does not connect the two sides of the sheet material. The transverse heat seals 304 are equally-spaced apart along the longitudinal length of web 300. This region 307 is sealed upon inflation of each air pillow 301. In one alternative embodiment, the transverse heat seal 304 may extend up to the upper edge 309. In another alternative embodiment, the transverse heat seal 304 may extend up to the upper edge 309 and across a portion of the space in region 307 above air chamber 308, leaving a narrower opening for the insertion air into air chamber 308 during inflation. Perforations 303 may be included within the transverse heat seal 304 area to allow easy separation of each air pillow 301 after inflation thereof. In an alternative embodiment, each of the air chambers 308 may include a plurality of separate channels in which gas may be held in the manner of bubble wrap.

Referring to FIG. 3B, a portion of an uninflated web 350 is shown after formation of the uninflated air pillows using two mating rolls of sheet material, each ply of web 350 formed from a separate roll of sheet material. In this embodiment, an additional heat seal 310 is provided between a lower edge 312 mating the two separate sheets and a line 311, sealing the two sheets together to form a lower seal 314 for each air chamber 308.

The embodiments shown in FIGS. 3A and 3B both include a completely open upper side. As one of ordinary skill in the art will readily understand, the opening into the formed but unfilled air pocket may be completely open or only partially open (depending on the type of machine used to fill the air pillows for example). Likewise, although the opening is shown on an upper edge in FIGS. 3A and 3B, the precise location of the opening may also be moved to a lower edge or even a transverse edge via a channel in a transverse heat seal. Each unformed air pillow has four edges, three of the edges completely sealed and a fourth edge that is either open or partially sealed.

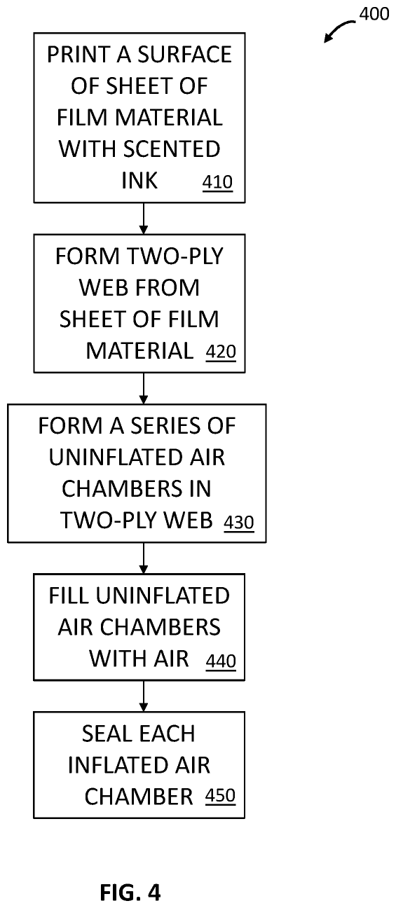

Referring now to FIG. 4, a flowchart is shown of the method of forming air pillows filled with scented air. First, at step 410, a scented material (e.g., a scented ink) is applied (e.g., by printing) to a first surface of a sheet of film material.

Next, at step 420, a two-ply web of film material is formed. In one embodiment, the two-ply web of film is formed from the sheet of film material by folding the sheet of film material along a longitudinal center axis thereof with the first surface of the sheet of film material becoming an inner surface of the two-ply web of film material. In this manner, the two-ply web of film material has an upper longitudinal edge formed from unconnected longitudinal edges of the sheet of the film material and a lower longitudinal edge formed from the fold along the longitudinal center axis of the sheet of film material. In an alternative embodiment, the two-ply web of film material is formed by mating the first sheet of film material to a second sheet of film material. In this embodiment, the first surface of the first sheet of film material is positioned to be an inner surface of the two-ply web of film material. The two-ply web has an upper longitudinal edge formed from unconnected upper longitudinal edges of the first and second sheets of the film material and a lower longitudinal edge formed from unconnected lower longitudinal edges of the first and second sheets of the film material.

Thereafter, at step 430, a series of uninflated air chambers is formed in the two-ply web of film material. Each of the uninflated air chambers is bounded transversely by equally-spaced apart transverse heat seals in the two-ply web of film material. In the first embodiment, each of the uninflated air chambers is bounded at a bottom edge by the fold along the longitudinal center axis of the sheet of film material. In the second embodiment, each of the uninflated air chambers is bounded at a bottom edge by a longitudinal heat seal along the lower longitudinal edge. Each of the uninflated air chambers is bounded at an upper edge by the open upper longitudinal edge. Each of the uninflated air chambers includes scented ink on an interior surface thereof.

Next, at step 440, one or more of the uninflated air chambers is filled with air. Finally, at step 450, the upper edge of each inflated air chamber is sealed with a heat seal. Each of the inflated sealed air chambers forms an air pillow that includes scented air because the scented ink on the inner surface of each of the inflated air chambers evaporates upon inflation thereof.

Although the present disclosure has been particularly shown and described with reference to the preferred embodiments and various aspects thereof, it will be appreciated by those of ordinary skill in the art that various changes and modifications may be made without departing from the spirit and scope of the disclosure. It is intended that the appended claims be interpreted as including the embodiments described herein, the alternatives mentioned above, and all equivalents thereto.

What is claimed is:

1. A method of forming an air pillow filled with scented air, comprising the steps of:
applying a scented material to a first surface of a sheet of film material;
forming a two-ply web of film material from the sheet of film material by folding the sheet of film material along a longitudinal center axis thereof, the first surface of the sheet of film material being an inner surface of the two-ply web of film material, the two-ply web of film material having an upper longitudinal edge formed from unconnected longitudinal edges of the sheet of the film material and a lower longitudinal edge formed from the fold along the longitudinal center axis of the sheet of film material;
forming a series of uninflated air chambers in the two-ply web of film material, each of the uninflated air chambers having four edges, each of the uninflated air chambers bounded at a first edge by the fold along the longitudinal center axis of the sheet of film material, bounded at a second edge and a third edge by heat seals in the two-ply web of film material, and bounded at a fourth edge by an open edge or a partially heat sealed edge, each of uninflated air chambers thereby including scented ink on an interior surface thereof; and
sequentially inflating one or more of the uninflated air chambers with air and sealing the open edge or partially sealed edge thereof with a heat seal, each of the sealed inflated air chambers forming an air pillow, whereby the scented ink on the inner surface of each of the inflated air chambers evaporates such that the air within each air pillow becomes scented.

2. The method of claim 1, wherein the step of applying a scented material to a first surface of a sheet of film material comprises applying a continuous pattern along a longitudinal length of the sheet of film material.

3. The method of claim 2, wherein the scented material is a scented ink and wherein the scented ink is applied to the first surface by printing the scented ink thereon.

4. The method of claim 1, wherein the step of applying a scented material to a first surface of a sheet of film material comprises applying a discontinuous pattern of elements along a longitudinal length of the sheet of film material.

5. The method of claim 4, wherein the scented material is a scented ink and wherein the scented ink is applied to the first surface by printing the scented ink thereon.

6. A method of forming an air pillow filled with scented air, comprising the steps of:
applying a scented material to a first surface of a first sheet of film material;
forming a two-ply web of film material by mating the first sheet of film material to a second sheet of film material, the first surface of the first sheet of film material being an inner surface of the two-ply web of film material, the two-ply web of film material having an upper longitudinal edge formed from unconnected upper longitudinal edges of the first and second sheets of the film material and a lower longitudinal edge formed from unconnected lower longitudinal edges of the first and second sheets of the film material;
forming a series of uninflated air chambers in the two-ply web of film material, each of the uninflated air chambers having four edges, each of the uninflated air chambers bounded at a first edge, a second edge, and a third edge by heat seals in the two-ply web of film material, and bounded at a fourth edge by an open edge or a partially heat sealed edge, each of the uninflated air chambers thereby including scented ink on an interior surface thereof; and
sequentially inflating one or more of the uninflated air chambers with air and sealing the open edge or partially sealed edge thereof with a heat seal, each of the sealed inflated air chambers forming an air pillow, whereby the scented ink on the inner surface of each of inflated air chambers evaporates such that the air within each air pillow becomes scented.

7. The method of claim 6, wherein the step of applying a scented material to a first surface of a sheet of film material comprises applying a continuous pattern along a longitudinal length of the sheet of film material.

8. The method of claim 7, wherein the scented material is a scented ink and wherein the scented ink is applied to the first surface by printing the scented ink thereon.

9. The method of claim 6, wherein the step of applying a scented material to a first surface of a sheet of film material comprises applying a discontinuous pattern of elements along a longitudinal length of the sheet of film material.

10. The method of claim 9, wherein the scented material is a scented ink and wherein the scented ink is applied to the first surface by printing the scented ink thereon.

11. A web of uninflated scented air cushions, comprising:
a sheet of film material having a scented material applied to a first surface thereof and formed into a two-ply web of film material by folding the sheet of film material along a longitudinal center axis thereof, the first surface of the sheet of film material being an inner surface of the two-ply web of film material, the two-ply web of film material having an upper longitudinal edge formed from unconnected longitudinal edges of the sheet of the film material and a lower longitudinal edge formed from the fold along the longitudinal center axis of the sheet of film material, the two-ply web of material having a series of uninflated air chambers, each of the uninflated air chambers having four edges, each of the uninflated air chambers bounded at a first edge by the fold along the longitudinal center axis of the sheet of film material, bounded at a second edge and a third edge by heat seals in the two-ply web of film material, and bounded at a fourth edge by an open edge or a partially heat sealed edge, each of uninflated air chambers thereby including scented ink on an interior surface thereof.

12. The web of claim 11, wherein the scented material is applied in a continuous pattern along a longitudinal length of the sheet of film material.

13. The web of claim 12, wherein the scented material is a scented ink and wherein the scented ink is applied to the first surface by printing the scented ink thereon.

14. The web of claim 11, wherein the scented material is applied to the first surface by applying a discontinuous pattern of elements along a longitudinal length of the sheet of film material.

15. The web of claim 14, wherein the scented material is a scented ink and wherein the scented ink is applied to the first surface by printing the scented ink thereon.

16. A web of uninflated scented air cushions, comprising:
a sheet of film material having scented material applied to a first surface thereof and formed into a two-ply web of film material by mating the first sheet of film material to a second sheet of film material, the first surface of the first sheet of film material being an inner surface of the two-ply web of film material, the two-ply web of film material having an upper longitudinal edge formed from unconnected upper longitudinal edges of the first and second sheets of the film material and a lower longitudinal edge formed from unconnected lower longitudinal edges of the first and second sheets of the film material, the two-ply web of material having a series of uninflated air chambers, each of the uninflated air chambers having four edges, each of the uninflated air chambers bounded at a first edge, a second edge, and a third edge by heat seals in the two-ply web of film material, and bounded at a fourth edge by an open edge or a partially heat sealed edge, each of uninflated air chambers thereby including scented ink on an interior surface thereof.

17. The web of claim 16, wherein the scented material is applied in a continuous pattern along a longitudinal length of the sheet of film material.

18. The web of claim 17, wherein the scented material is a scented ink and wherein the scented ink is applied to the first surface by printing the scented ink thereon.

19. The web of claim 16, wherein the scented material is applied to the first surface by applying a discontinuous pattern of elements along a longitudinal length of the sheet of film material.

20. The web of claim 19, wherein the scented material is a scented ink and wherein the scented ink is applied to the first surface by printing the scented ink thereon.
